(19) United States
(12) Patent Application Publication
(10) Pub. No.: US 2014/0255358 A1
(43) Pub. Date: Sep. 11, 2014

(54) GENERATION OF IMMUNOSUPPRESSIVE MYELOID CELLS USING PGE2

(71) Applicant: Pawel Kalinski, Wexford, PA (US)

(72) Inventors: Pawel Kalinski, Wexford, PA (US); Ravikumar Muthuswamy, Monroeville, PA (US); Natasa Obermajer, Scopolijeva (SI)

(21) Appl. No.: 14/348,320

(22) PCT Filed: Oct. 1, 2012
(86) PCT No.: PCT/US12/58356

§ 371 Date: Mar. 28, 2014

Related U.S. Application Data
(60) Provisional application No. 61/541,809, filed on Sep. 30, 2011.

(51) Int. Cl. A61K 38/19; A61K 31/201; A61K 35/14

(52) U.S. Cl. CPC A61K 38/195; A61K 35/15; A61K 31/201
USPC 424/93.7; 514/21.2

(57) ABSTRACT

Therapies effective for the treatment and prevention of autoimmune diseases, chronic inflammatory diseases, transplant rejection or Graft versus Host Disease (GvH), using prostaglandin (PG), alternative agonists of PG receptors, EP2 or EP4, or other activators of adenylate cyclase/cAMP/PKA/CREB signaling pathway are disclosed herein. These methods include the administration of a therapeutically effective amount of myeloid cells pre-treated ex vivo with the above-mentioned factors, or the in vivo administration of such agents in combination with the factors attracting myeloid precursor cells, such as myeloid cell-attracting chemokines or their inducers. Such therapies can be applied for the prevention or treatment of autoimmune diseases, spontaneous and specific pathogen-induced inflammatory diseases (including some infectious diseases), premalignant and malignant lesions, and for prevention and treatment of transplant rejection and GvH.

### STATEMENT OF PRIORITY

This claims the benefit of U.S. Provisional Application No. 61/541,809, filed Sep. 30, 2011, which is incorporated by reference herein.

### STATEMENT OF GOVERNMENT SUPPORT

This invention was made with United States government support pursuant to grant 1PO1 CA132714 from the National Institutes of Health; the United States government has certain rights in the invention.

### FIELD

This is related to the field of treatment or prevention of autoimmune diseases, allergies and inflammation, as well as to treat or prevent transplant rejection and transplantation-associated disorders, including graft versus host disease (GvH).

### BACKGROUND

PGE₂ and other Prostaglandins. Prostaglandins (PGs) are small-molecule derivatives of arachidonic acid, produced by cyclooxygenases (constitutively active COX1 and inducible COX2) and prostaglandin synthases, with a relatively minor contribution of the isoprostane pathway. Prostaglandin E₂ (PGE₂), (and to a lesser extend Prostaglandin D2), is the main product of cyclooxygenases in most physiological conditions, regulated by the local balance between its COX2-regulated synthesis and 15-PGDH-regulated degradation (see FIG. 7). The receptors for PGE₂ (EP1-EP4) are present on multiple cell types, reflecting the ubiquitous functions of PGE₂, which span nociception and other aspects of neuronal signaling, hematopoiesis, regulation of blood flow, renal filtration and blood pressure, regulation of mucosal integrity, vascular permeability and smooth muscle function. PGE₂ is generally recognized as a mediator of active inflammation, promoting local vasodilatation and local attraction and activation of neutrophils, macrophages, and mast cells at early stages of inflammation, its ability to promote the induction of suppressive IL-10- and to directly suppress the production of multiple pro-inflammatory cytokines allows it to limit nonspecific inflammation, promoting the immune suppression associated with chronic inflammation and cancer. While PGE₂ can promote the activation, maturation and migration of dendritic cells (DC; see below), the central cells during the development of antigen-specific immunity, it has been widely demonstrated to suppress both innate and antigen-specific immunity at multiple molecular and cellular levels, earning PGE₂ the paradoxical status of a pro-inflammatory factor with immunosuppressive activity.

While PGE₂ inhibitors, such as steroids (inhibitors of AA release) and non-steroid anti-inflammatory drugs (NSAIDs; blockers of COX 1/2 or COX2 function) represent some of the most-common and effective pharmaceutical agents, realizing the full potential of PGE₂ targeting in the treatment of chronic infections, inflammation and cancer, is restricted by the complex pattern of PGE₂-mediated immunoregulation and our still-incomplete understanding of the key mechanisms and targets of PGE₂-mediated immunoregulation.

Regulation of PGE₂ production. PGE₂ synthesis involves phospholipase A2 (PLA2), cyclooxygenases (COX1 and COX2) that convert arachidonic acid into prostaglandin H₂ (PGH₂), and prostaglandin E synthases (PGES), responsible for the final synthesis of PGE₂.

PGE₂ degradation. PGE₂ is relatively stable in vitro although its decay is accelerated by albumin. In contrast, PGE₂ has a very rapid turnover rate in vivo and is rapidly eliminated from tissues and circulation. The rate of PGE₂ degradation in vivo in individual tissues is controlled by 15-hydroxyprostaglandin dehydrogenase (15-PGDH). The suppression of 15-PGDH activity observed in many forms of cancer or UV-irradiated skin, known to be PGE₂-rich and immunosuppressive environments, suggest that in addition to the rate of PGE₂ synthesis, also the rate of PGE₂ decay may be subject to regulation in physiological conditions and pathology, and may constitute a potential target of immunomodulation. In accord with such possibility, it was recently shown that apoptotic cancer cells can modulate the prostanoid production in macrophages by up-regulating COX2 and microsomal prostaglandin E synthase-1 (mPGES1), while down-regulating the 15-PGDH. Moreover, the deactivation of 15-PGDH has been shown responsible for the resistance of premalignant colon lesions to celecoxib.

PGE₂ receptors and signaling pathways: Regulation of PGE₂ responsiveness. The heterogeneous effects of PGE₂ are reflected by the existence of four different PGE₂ receptors, designated EP1, EP2, EP3 and EP4, with an additional level of functional diversity resulting from multiple splice variants of EP3 that exists in at least 8 forms in humans and 3 forms in mice.

EP3 and EP4 represent high affinity receptors, while EP1 and EP2 require significantly higher concentrations of PGE₂ for effective signaling. The signaling through the two Gₛ-coupled receptors, EP2 and EP4, is mediated by the adenylate cyclase-triggered cAMP/PKA/CREB pathway, mediating the dominant aspects of the anti-inflammatory and suppressive activity of PGE₂. Despite their similar nominal functions, the signaling by EP2 and EP4 is triggered by different concentrations of PGE₂ and differs in duration. EP4 signaling is rapidly desensitized following its PGE₂ interaction, while EP2 is resistant to ligand-induced desensitization, implicating its ability to mediate PGE₂ functions over prolonged periods of time, and at later time-points of inflammation. While EP2 is believed to signal in a largely cAMP-dependent fashion, EP4 also activates the PI3K-dependent ERK1/2 pathway. However, both EP2 and EP4 have been shown to activate the GSK3/β-catenin pathway.

EP1 and high affinity EP3 are not coupled to Gₛ and lack cAMP-activating functions. Most of the splice variants of EP3 represent Gᵢ-coupled PGE₂ receptors capable of inhibiting cAMP, although at least some of them can also exist in a Gₛ-coupled form capable of cAMP activation, with different sensitivities to ligand-induced desensitization. The mode of signaling via EP1 remains relatively unclear, but involves calcium release.

Additional flexibility of the PGE₂ receptor system results from different sensitivity of the individual receptors to regulation by PGE₂ and additional factors. Expression of EP2 and the resulting responsiveness to PGE₂ can be suppressed by hyper-methylation, as seen in patients with idiopathic lung fibrosis. These observations raise the possibility that, in addition to the regulation of PGE₂ production and its degradation, the regulation of PGE₂ responsiveness at the level of expression of individual PGE₂ receptors can also contribute to the pathogenesis of human disease and be exploited in their therapy.

Dendritic cells (DC), suppressive macrophages and myeloid-derived suppressor cells (MDSC). Dendritic cells (DCs) are key initiators and regulators of immune responses. Suppression of endogenous DCs' functions has been shown to contribute to cancer progression, therapeutic targeting of DCs to suppress their function has been shown beneficial in mouse models of autoimmunity or transplantation.

In contrast to DCs, suppressive macrophages, myeloid-derived suppressor cells (MDSCs), or other types of suppressive myeloid cells, all suppress the ability of CD8⁺ T cells to mediate effective responses against cancer cells, but can be beneficial in controlling autoimmune phenomena or transplant rejection. MDSCs express CD34, common myeloid marker CD33, macrophage/DC marker CD11b, and IL4Rα (CD124), but lack expression of the lineage (Lin) markers of DC and other mature myeloid cells. Human MDSCs are defined as CD33⁺Lin⁻HLA-DR^{-/low} or CD33⁺CD14⁻HLA-DR⁻, with recent studies demonstrating a CD14⁺CD11b⁺HLA-DR^{low} phenotype of monocytic MDSCs in melanoma, prostate cancer, gastrointestinal malignancies, hepatocellular carcinoma and glioblastoma, in addition to a CD15⁺ population of neutrophil-related immature (i)MDSCs of similar biologic activity present in peripheral blood. MDSCs and other myeloid suppressive cells, express high levels of immunosuppressive factors, such as indoleamine dioxygenase (IDO), IL-10, arginase, inducible nitric oxide synthase (iNOS, NOS2), nitric oxide (NO), and reactive oxygen species (ROS), and use these molecules to suppress T-cell responses, while their induction of NK cell anergy and reduced cytotoxicity is arginase-independent but depends on TGFβ₁. Besides, PD-L1/B7-H1, induced on MDSCs in the tumor microenvironment, suppresses antigen-specific immunity via interaction with regulatory T cells (T_{reg}) and reduces tumor clearance via enhanced T cell IL-10 expression and reduced IFN-γ production.

Molecular pathways involved in negative regulation of DC function remain largely unknown, however. Upregulation of myeloid cells expressed inhibitory receptors immunoglobulin-like transcript receptors (ILT)-3 and ILT-4, that can negatively regulate activation of DCs, induces T-cell tolerance.

The development of functional MDSCs requires the inhibition of development of immunostimulatory APCs and concomitant induction of suppressive functions. Such factors as granulocyte macrophage colony stimulating factor (GM-CSF), interleukin-6 (IL-6), or vascular endothelial growth factor (VEGF) promote expansion of iMCs. An additional signal is required for the upregulation of MDSC-associated immunosuppressive factors and establishment of their immunosuppressive function.

The absence of defined minimal requirements for the development of MDSCs and other myeloid suppressive cells raises obvious obstacles to the development of effective promote the development of myeloid suppressive cells for therapeutic purposes. PGE₂, a proinflammatory molecule produced by cancer cells, stroma, and infiltrating myeloid cells, can promote final maturation of the already developed DCs, increasing their stimulatory function. However, the presence of PGE₂ already at early stages of DC development, was shown to suppresses the differentiation of human monocytes into functional Th1-inducing CD1a⁺DCs, although the resulting cells have not been shown yet to have suppressive activity. PGE₂ has also been shown to enhance the numbers of MDSCs in mouse models and the expression of arginase I in human MDSCs. Despite their diverse character and functions, numerous other factors implicated in MDSC development share the ability to induce COX2 expression and PGE₂ production. As discussed here, COX2/PGE₂ expression represents the critical minimal requirement needed for the development of functionally stable myeloid suppressive cells that are functionally similar to MDSCs.

### SUMMARY

Therapies effective for the treatment and prevention of autoimmunity, inflammation, transplant rejection, GvH and other diseases are disclosed herein. These methods include the administration of a therapeutically effective amount of either myeloid cells exposed ex vivo to a prostaglandin, prostaglandin analog or alternative activator of the cAMP-signaling pathway, or the combined in vivo administration of a prostaglandin, prostaglandin analog or alternative activator of the cAMP-signaling pathway, jointly with an attractant of myeloid cells.

We describe a simple and clinically-compatible method of generating large numbers of immunosuppressive cells with the characteristics of MDSCs from myeloid precursors, including peripheral blood precursor cells (FIGS. 1-6), using exogenous prostaglandin E2 (PGE₂), the factor known to signal vial EP2, EP2, EP3, and EP4 receptors and to activate the adenylate cyclase-triggered cAMP/PKA/CREB signaling pathway (see FIG. 7). We observed that PGE₂ induces monocyte expression of COX2, blocking their differentiation into CD1a⁺ DCs and induces the expression of IDO1, IL4Rα, NOS2 and IL-10, potent immunosuppressive factors. The establishment of positive feedback loop between prostaglandin E₂ (PGE₂) and COX2, the key regulator of PGE₂ synthesis, represents the determining factor in redirecting the development of CD1a⁺ DCs to CD14⁺CD33⁺CD34⁺ monocytic suppressive cells. Addition of PGE₂ to GM-CSF/IL-4-supplemented monocyte cultures is sufficient to induce the CTL-suppressive function, the production of multiple immunosuppressive mediators and the phenotype typical of suppressive cells. In addition to PGE₂, also EP2- and EP4-agonists, but not EP3/1 agonists induce the production of suppressive factors and the CTL-inhibitory function, indicating that other activators of EP2 and EP2-induced adenylate cyclase/cAMP/PKA/CREB signaling pathway can be used to promote the development of suppressive cells. Hence, PGE₂, EP2 and EP4 agonists (or factors enhancing their expression), mediators of their downstream signaling, or inhibitors of PGE₂ degradation can be used to generate large numbers of myeloid suppressive cells for the immunotherapy of autoimmune diseases, spontaneous and specific pathogen-induced inflammatory diseases including some infectious diseases), development of premalignant and malignant lesions, for certain forms of infertility, to accelerate wound healing, and for prevention and treatment of transplant rejection.

### DETAILED DESCRIPTION

Therapies effective for the treatment and prevention of autoimmunity, inflammation, transplant rejection, GvH and other diseases are disclosed herein. These methods include the administration of a therapeutically effective amount of either myeloid cells exposed ex vivo to a prostaglandin, prostaglandin analog or alternative activator of the cAMP-signaling pathway, or the combined in vivo administration of a prostaglandin, prostaglandin analog or alternative activator of the cAMP-signaling pathway, jointly with an attractant of myeloid cells.

### Terms

I. Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Chemokines: Immune chemoattractants inducing the migration of immune cells towards its source (against the gradient). SDF1/CXCL12 is an example of a chemokine. It binds CXCR4, expressed on suppressive myeloid cells and promotes their accumulation in tissues.

Myeloid cell attracting chemokines include CCL2, CCL3, CCL4, CCL5, CCL6, CCL7, CCL8, CCL12, CCL3, CL15, CCL16, CCL20, CCL23, CXCL14 and CX3CL1.

Preventing, treating or ameliorating a disease: “Preventing” a disease refers to inhibiting the full development of a disease, or delaying the development of the disease. “Treating” refers to a therapeutic intervention that ameliorates a sign or symptom of a disease or pathological condition after it has begun to develop. “Ameliorating” refers to the reduction in the number or severity of signs or symptoms of a disease, such as autoimmunity.

Prostaglandins (PGs) and their immunosuppressive functions. Prostaglandins (PGs) are members of a group of lipid compounds derived enzymatically from fatty acids. Prostaglandins, together with the thromboxanes and prostacyclins, form the prostanoid class of fatty acid derivatives, a subclass of eicosanoids. Prostaglandins, particularly of the E series (PGEs) and their analogs show multiple immunosuppressive functions mediated mostly by two receptors, EP2 and EP4, that promote activation of adenylate cyclase and cAMP accumulation within responding cells.

(11 alpha,13E,15S)-11,15-Dihydroxy-9-oxoprost-13-en-1-oic acid (PGE(1)); (5Z,11 alpha,13E,15S)-11,15-dihydroxy-9-oxoprosta-5,13-dien-1-oic acid (PGE(2)); and (5Z,11 alpha,13E,15S,17Z)-11,15-dihydroxy-9-oxoprosta-5,13,17-trien-1-oic acid (PGE(3)) represent three E class PGs of the six naturally occurring prostaglandins. Clinically-used prostaglandins and synthetic prostaglandin analogs include Alprostadil, Arbaprostil, Misoprostol, Enprostil, and Rioprostil, and 16,16-Dimethylprostaglandin E2.

PG production. The process of PG synthesis involves phospholipase A2 (PLA2) family members, that mobilize arachidonic acid from cellular membranes, cyclooxygenases (constitutively-active COX1 and inducible COX2) that convert arachidonic acid into prostaglandin H₂ (PGH₂), and prostaglandin E synthase (PGES), needed for the final formulation of PGE₂. While the rate of PGE₂ synthesis and the resulting inflammatory process can be affected by additional factors, such as local availability of AA, in most physiologic conditions, the rate of PGE₂ synthesis is controlled by local expression and activity of COX2.

PGE₂ degradation. The rate of PGE₂ degradation is controlled by 15-hydroxyprostaglandin dehydrogenase (15-PGDH), suggesting that in addition to the rate of PGE₂ synthesis, also the rate of PGE₂ decay constitutes a target for immuno-modulation.

PGE₂ responsiveness. Four different PGE₂ receptors are EP 1, EP2, EP3 and EP4. The signaling through the two Gₛ-coupled receptors, EP2 and EP4, is mediated by the adenylate cyclase-triggered cAMP/PKA/CREB pathway, mediating the dominant aspects of the anti-inflammatory and suppressive activity of PGE₂. While EP2 is believed to signal in a largely cAMP-dependent fashion, EP4 also activates the PI3K-dependent ERK1/2 pathway. However, both EP2 and EP4 have been shown to activate the GSK3/β-catenin pathway.

The expression of EP2 and the resulting responsiveness to PGE₂ can be suppressed by hyper-methylation, as observed in patients with idiopathic lung fibrosis. These observations raise the possibility that, in addition to the regulation of PGE₂ production and its degradation, the regulation of PGE₂ responsiveness at the level of expression of individual PGE₂ receptors can also contribute to the pathogenesis of human disease and be exploited in their therapy. In support of this possibility, the use of synthetic inhibitors, preferentially affecting EP2, EP3, or EP4 signaling, allow for differential suppression of different aspects of PGE₂ activity.

Prostaglandin (PG) Synthesis Inhibitors: Factor which inhibit the synthesis of PGs in general or the synthesis of a specific type of PGs. PG synthesis inhibitors include nonselective inhibitors of COX-1 and COX-2, the two key enzymes in the PG synthesis pathway, and selective inhibitors of COX-2, which are believed to be more specific to COX-2 and less toxic. The examples of non-selective PG inhibitors include aspirin, indomethacin, or ibuprofen (Advil, Motrin). The examples of COX-2-selective inhibitors include Celecoxib (Celebrex) and rofecoxib (Vioxx). The example of COX-1-specific inhibitor is sulindac (Clinoril). Other drugs that suppress prostaglandin synthesis include steroids (example: hydrocortisone, cortisol, prednisone, or dexamethasone) and acetaminophen (Tylenol, Panadol), commonly used as anti-inflammatory, antipyrrhetic and analgesic drugs. Examples of the most commonly used selective COX2 inhibitors include celecoxib, alecoxib, valdecoxib, and rofecoxib.

Examples of the most commonly used non-selective COX 1 and COX2 inhibitors include: acetylsalicylic acid (aspirin) and other salicylates, acetaminophen (Tylenol), ibuprofen (Advil, Motrin, Nuprin, Rufen), naproxen (Naprosyn, Aleve), nabumetone (Relafen), or diclofenac (Cataflam).

Prostaglandin (PG) Signaling Pathways. Prostaglandins signal through numerous receptors, with the key immunosuppressive effects being mediated by the activation of adenylate cyclase, the resulting elevation of the intracellular cyclic (c)AMP, PKA and the downstream activation of the PKA/CREB pathway.

Another level of interference with the PG responsiveness includes the interference with their bringing to PG receptors. In case of PGE2, the two key cAMP-activating receptors are EP2 and EP4, for which a number of specific inhibitors exist.

The increase of cAMP levels induced by prostaglandings or other factors can be prevented by phosphodiesterases (PDEs; currently known 6 types, PDE1-PDE5 and PDE10, which reduce the levels of intracellular cAMP). PDEs can be controlled by phoshodiestherase inhibitors, which include such substances as xanthines (caffeine, aminophylline, IBMX, pentoxyphylline, theobromine, theophylline, or paraxanthine), which all increase the levels of intracellular cAMP, and the more selective synthetic and natural factors, including vinpocetine, cilostazol, inamrinone, cilostazol, mesembrine, rolipram, ibudilast, drotaverine, piclamilast, sildafenil, tadalafil, verdenafil, or papaverine.

Furthermore, interference with PGE2 signalling (or with the signaling of other cAMP-elevating factors, such as histamine, of beta-adrenergic agonists) can be achieved by the inhibition of downstream signals of cAMP, such as PKA or CREB.

The key suppressive and tumor-promoting effects of prostaglandins are mediated by the activation of adenylate cyclase, the resulting elevation of the intracellular cyclic (c)AMP, PKA and the downstream activation of the PKA/CREB pathway.

Pro-inflammatory cytokines: Proinflammatory cytokines known to inducer the production of chemokines include GM-CSF, M-CSF, tumor necrosis factor alpha (TNFα) and TNF-beta (TNFβ), Interleukins (ILs), including IL-1α and IL-1β, or IL-6, and interferons (IFNs), including IFNα, IFNβ an IFNγ. Chemokines can be also induced by lipid mediators of inflammation, including prostaglandins and leukotriens or nominally non-cytokine endogenous alarm signals released from damaged cells, such as HMGB1 or uric acid.

Therapeutically effective amount: An amount of a therapeutic agent (such as PGE2 or an agent that increases cAMP levels in target cells) that alone, or together with one or more additional therapeutic agents, induces the desired response, such the induction of immunosuppressive factors in target cells. Ideally, a therapeutically effective amount provides a therapeutic effect without causing a substantial cytotoxic effect in the target cells or in a subject. The preparations disclosed herein are administered in therapeutically effective amounts.

In general, an effective amount of a composition administered to a human subject will vary depending upon a number of factors associated with that subject, for example the overall health of the subject, the condition to be treated, or the severity of the condition. An effective amount of a composition can be determined by varying the dosage of the product and measuring the resulting therapeutic response, such as the increase in the production of immunosuppressive molecules in target cells, including IDO, arginase, NO, VEGF or IL-10, or the increase of the immunosuppressive activity of target cells, or the resulting suppression of T cell- and NK cell responses. Any agent can be administered in a single dose, or in several doses, as needed to obtain the desired response. However, the effective amount can be dependent on the source applied, the subject being treated, the severity and type of the condition being treated, and the manner of administration.

Toll-like Receptors (TLR): A family of receptors which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity. There are a total of total of 13 mammalian TLRs, including nine (TLR1-9) that have been extensively studied and are known to activate the NF-kB pathway.

Toll-like receptor 3 (TLR3): A member of the Toll-like receptor (TLR) family. Its amino acid sequence of is shown in NCBI accession number NP_{—}003256, as of Jan. 2, 2009, the disclosure of which is incorporated herein by reference. TLR3 is a member of the Toll-like receptor (TLR). This receptor is most abundantly expressed in placenta and pancreas, and is restricted to the dendritic subpopulation of the leukocytes. It recognizes dsRNA associated with viral infection, and induces the activation of NF-κB and the production of type I interferons.

TLR3 Agonists: A TLR3 agonist can be selected from any suitable agent that activates TLR3 and/or the subsequent cascade of biochemical events associated with TLR3 activation in vivo. A compound can be identified as an agonist of TLR3 if performing the assay with that compound results in at least a threshold increase of some biological activity known to be mediated by TLR3. Conversely, a compound may be identified as not acting as an agonist of TLR3 if, when used to perform an assay designed to detect biological activity mediated by TLR3, the compound fails to elicit a threshold increase in the biological activity. Assays employing HEK293 cells transfected with an expressible TLR3 structural gene may use a threshold of for example, at least a three-fold increase in a TLR3-mediated biological activity (such as NF-KB activation) when the compound is provided at a concentration of, for example, from about 1 μM to about 10 μM for identifying a compound as an agonist of the TLR3 transfected into the cell. However, different thresholds and/or different concentration ranges may be suitable in certain circumstances.

A TLR3 agonist can be an agonistic antibody, an agonistic fragment of such antibodies, a chimeric version of such antibodies or fragment, or another active antibody derivative, TLR3 agonist antibodies useful in this invention may be produced by any of a variety of techniques known in the art.

Examples of a TLR3 agonist is AMPLIGEN™ (Hemispherx, Inc.,), a dsRNA formed by complexes of polyriboinosinic and polyribocytidylic/uridylic acid, Polyadenur (Ipsen), is STEALTH™ RNAi (commercially available from Invitrogen, Carlsbad, Calif. USA) or stabilized dsRNA poly-ICLC (Hiltonol, produced by Oncovir).

Ligands of alternative Toll-like receptors (TLRs 1-9) also known to induce the production of chemokines that attract myeloid cells: There have been a total of 13 TLRs identified in mammals, including nine (TLR1-9) that have been expensively studied and are known to induce chemokine production.

Activated TLRs recruit adapter molecules within the cell cytoplasm to initiate signal transduction. At least four adapter molecules, MyD88, TIRAP (Mal), TRIF, and TRAM are known to be involved in signaling.

TLR signaling is divided into two distinct signaling pathways, the MyD88-dependent and TRIF-dependent pathway. The MyD88-dependent response occurs on dimerization of the TLR receptor, and is utilized by every TLR except TLR3. The primary effect of MyD88 activation is the activation of NF-κB. MyD88 (a member of TIR family) recruits IRAM kinases IRAK 1, IRAK 2, and IRAK 4. IRAK kinases phosphorylate and activate the signaling protein TRAF6, which in turn polyubiquinates the protein TAK1, as well as itself in order to facilitate binding to IKKβ. On binding, TAK1 phosphorylates IKKβ, which then phosphorylates IκB causing its degradation and allowing NF-κB to enter the cell nucleus and activate transcription.

Both TRL3 and TRL4 utilize the TRIF-dependent pathway, which is triggered, respectively, by dsRNA and LPS. For TRL3, dsRNA leads to activation of the receptor, recruiting the adaptor TRIF. TRIF activates the kinases TBK1 and RIP1. The TRIF/TBK1 signaling complex phosphorylates IRF3, promoting its entry into the nucleus and production of type I IFNs. The activation of RIP1 causes the polyubiquination and activation of TAK1 (joint pathway with MyD88 signaling and NFκB transcription, similar to the MyD88-dependent pathway of other TLR signaling.

In addition to TLR ligands, production of chemokines and prostaglandins can be also induced by Notch ligands.

### Compositions and Therapeutic Methods

Method are disclosed herein for preventing or treating inflammation, autoimmunity, transplant rejection and graft versus host disease (GvH). The methods include administering a therapeutically effective amount of agents that increase cAMP levels in the relevant cells and organs.

An amount of a therapeutic agent is considered effective if it together with one or more additional therapeutic agents, induces the desired response, such as decreasing the risk of developing a diseases, treating the disease, slowing down its progression, preventing its recurrence, or alleviating the signs and symptoms of the disease. In one example, it is an amount of an agent needed to prevent or delay the development of a disease, in a subject. Ideally, a therapeutically effective amount provides a therapeutic effect without causing a substantial cytotoxic effect in the subject. The preparations disclosed herein are administered in therapeutically effective amounts.

Compositions are provided that include one or more of the agents disclosed herein that are disclosed herein in a carrier. The compositions can be prepared in unit dosage forms for administration to a subject. The amount and timing of administration are at the discretion of the treating physician to achieve the desired purposes. The agent can be formulated for systemic or local administration. In one example, the agents are formulated for parenteral administration, such as intravenous administration.

The compositions for administration can include a solution of the agents of use dissolved in a pharmaceutically acceptable carrier, such as an aqueous carrier, or bio-compatible formulations of liposomes or other bio-compatible vesicles, or other slow release matrices and vehicles. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of the active component in these formulations can vary widely, and will be selected primarily based on fluid volumes, viscosities, body weight and the like in accordance with the particular mode of administration selected and the subject's needs.

A therapeutically effective amount of the agents of use will depend upon the severity of the disease and the general state of the patient's health. A therapeutically effective amount of the agent when administered to a subject that has autoimmunity, inflammation or transplantation-related symptoms is that which provides either subjective relief of a symptom(s) or an objectively identifiable improvement as noted by the clinician or other qualified observer. These compositions can be administered in conjunction with another chemotherapeutic agent, either simultaneously or sequentially.

The optimal activity of drugs frequently requires their prolonged administration, and in case of the combination administration of different drugs, it may require their administration in a specific sequence. Both of these requirements can be fulfilled by the application of controlled delivery systems, releasing one, three or more of the components of the treatment with similar or different kinetics, starting at the same time point or sequentially.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, N.Y., pp. 219-342 (1994); and Tice & Tabibi, Treatise on Controlled Drug Delivery, A. Kydonieus, ed., Marcel Dekker, Inc. New York, N.Y., pp. 315-339, (1992) both of which are incorporated herein by reference.

Polymers can be used for ion-controlled release of the compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### The Disclosure is Illustrated by the Following Non-Limiting Examples.

We observed that while monocytes constitutively express COX1, the PGE₂-exposed short-term cultured monocytes start expressing endogenous COX2 (FIG. 1A), indicating the ability of PGE₂ to initiate COX2-mediated positive feedback in differentiating DC precursors. Furthermore, exposure to PGE₂ also induced the expression of additional immunosuppressive factors: IDO1, NOS2, IL-10, or IL4Rα (FIG. 1B).

As shown in FIG. 2, the addition of PGE₂ to the GM-CSF and IL-4-supplemented cultures of differentiating monocytes, but not to the cultures of the already-developed immature CD1a⁺CD14⁻ DC (PGE₂-conditioned DCs, day 6), abolished the induction of CD1a⁺CD14⁻ DCs and promoted the development of CD1a⁻CD14⁺CD80⁻ CD83⁻ cells (FIGS. 2A-C). PGE₂ acts on four subtypes of G protein-coupled receptors designated EP1, EP2, EP3, and EP4, among which EP2 and EP4 signaling is coupled to rise in cAMP concentration. PGE₂-induced myeloid suppressive cells expressed all EP1-EP4 receptors and similar to the short-term-cultured monocytes, the monocytic suppressive cells developing in six-day-long PGE₂-supplemented cultures expressed high levels of COX2 (FIG. 2B), demonstrating the establishment of long-term PGE₂-COX2-mediated positive feedback loop in the myeloid suppressive cells. PGE₂-induced cells displayed suppressive phenotype, marked by the expression of inhibitory molecules ILT2, ILT3, ILT4 and PDL-1, which have been previously implicated in the suppressive functions of myeloid cells (FIG. 2A), and production of suppressive factors (FIGS. 2B) and suppressive functions (FIG. 2C).

Moreover, EP2 agonist Butaprost and EP4 agonist CAY10598, but not EP3/1 agonist Sulprostone, induced high levels of immunosuppressive factors (and markers of suppressive cells): arginase, IDO1, NOS2, IL-4Rα, IL10 and COX2 mRNA (FIG. 3I), indicating that the induction of myeloid suppressive cells involves both, EP2 and EP4.

Prompted by the key role of a PGE₂- in the reversal of DC generation and concomitant de novo induction of functional myeloid suppressive cells, we tested whether the stability of mature myeloid suppressive cells depends on the presence of IL-4.

As shown in FIG. 4, stability of the PGE₂-induced myeloid suppressive cells s depends on high dose of long term exposure of PGE₂, but is independent of the presence of IL-4. PGE₂ dose-dependently induced their production of IL-10, ARG-1, NOS2 and IDO1, whereas the induction of IL4Rα was less pronounced, but also significant (FIG. 4).

Yield of PGE₂-induced myeloid suppressive cells was similar as the yield of iDCs and TNF-a matured DCs (FIG. 5).

Collectively, these data indicate the minimal requirement for PGE₂, but not of IL-4 in mediating the induction of myeloid suppressive cells.

Our current data demonstrate that PGE₂ in differentiating monocytes is the necessary and sufficient factor in the redirecting of functional differentiation of monocytes towards myeloid suppressive cells.

These observations indicate that PGE₂, redirects the differentiation of myeloid cells towards myeloid suppressive cells (FIG. 6).

The ability of EP2- and EP4-, but not EP3/1-, agonists to reproduce PGE₂-induced effects demonstrates the key role of EP2 and EP4 in mediating the suppressive cell-promoting effects of PGE₂ and suggests additional targets for pharmacologic targeting. These data show that PGE₂ plays the key role of in the differentiation of myeloid suppressive cells, and that its action is mediated by EP4 or EP2 receptors, known activators of cAMP signaling.

The current observations contribute to the explanation of the minimal requirement in the mechanism of myeloid cell generation and the role of PGE₂- in this process. They explain the apparently multi-factorial mechanism of the induction of myeloid suppressive cells and provide clinically-feasible targets (COX2, EP2 and EP4) for counteracting immune suppression. They also provide for a system to generate large numbers of myeloid suppressive cells ex vivo, and, by analogy, in vivo, facilitating the development of additional myeloid suppressive cells targeting strategies for the prevention and treatment of autoimmunity, chronic inflammation, certain forms of cancer, and other diseases including transplant rejection and GvH.

Since PGE₂ and other EP2 and EP4 agonists are known to promote the production of CXCL4/SDF1 at the sites of inflammation and promote local accumulation of suppressive myeloid cells (that express CXCR4, receptor for CXCL12/SDF1), therapeutic administration of such suppressive cells is likely to be particularly effective when combined with systemic administration of PGE₂ and other EP2 and EP4 agonists, in order to direct the migration of myeloid suppressive cells to the sites of ongoing autoimmune or inflammatory reaction.

### BRIEF DESCRIPTION OF THE DRAWINGS

FIG. 1. The induction of endogenous COX2 and MDSC-associated suppressive factors in monocytes by PGE₂. (A) Expression of COX2 mRNA (A, left) and protein (A, right) levels in monocytes isolated from healthy blood donors is induced by synthetic PGE₂. Regulation of COX1 and COX2 expression by synthetic PGE₂ was analyzed after 6-10 h. (B) Induction of immunosuppressive factors arginase I, IL-10, NOS2, IDO1, IL4Rα by synthetic PGE₂. All data (panels A-B) were confirmed in at least 3 independent experiments. Bar graphs present data of a single representative experiment with different donors as mean±s.d. P<0.05 marked *; P<0.01 marked **; P<0.001 marked ***.

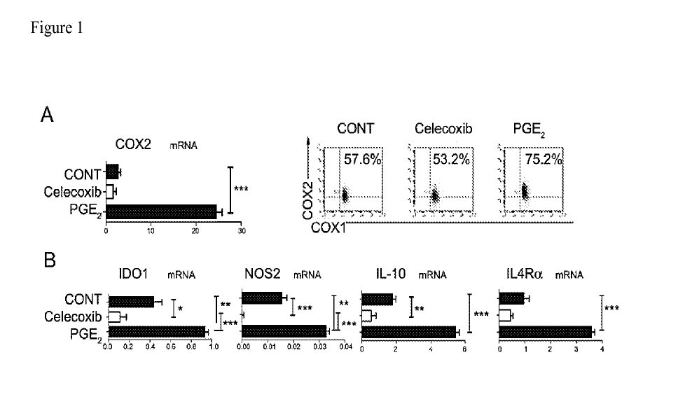

FIG. 2. PGE₂ redirects DC differentiation and induces CD14⁺CD33⁺CD34⁺ cells with the phenotype and function similar to monocytic MDSCs. (A) Phenotype of PGE₂-induced CD1a⁻CD14⁺CD80⁻CD83⁻ suppressive cells expressing inhibitory molecules ILT2, ILT3, ILT4, PDL-1, but not PDL-2. PGE₂-induced suppressive cells express E-prostanoid receptors (labeled with α-EP1-, α-EP3- sec.Alexa488 and α-EP2-, α-EP4-PE). (B) (top) Increased intracellular protein levels of immunosuppressive factors IDO1 and COX2 expression and IL-10 production in PGE₂-treated cells (PGE₂-induced suppressive cells) compared to control DCs after 6 days of culture (261 pg/ml for PGE₂-treated and 1.8 pg/ml for control cells). (bottom) Expression of immunosuppressive factors IL10, IDO1, IL4Rα and COX2 in PGE₂-induced suppressive cells. (C) Immunosuppressive effects of PGE₂-induced suppressive cells on allogeneic naïve CFSE-labeled CD8⁺ T cells primed by CD3/CD28 and stained for granzyme B. Left panel: Percentages indicate the fraction of proliferating granzyme B⁺ (marker of CTL status) CD8⁺ cells. Right panel: Percentage of proliferating CD8⁺ T cells in the presence of PGE₂-induced suppressive cells (PGE₂-d0) and PGE₂-conditioned DCs (PGE₂-d6). All data (panels A-C) were confirmed in at least 3 independent experiments. Bar graphs present data of a single representative experiment with different donors as mean±s.d. P<0.05 marked *; P<0.01 marked **; P<0.001 marked ***.

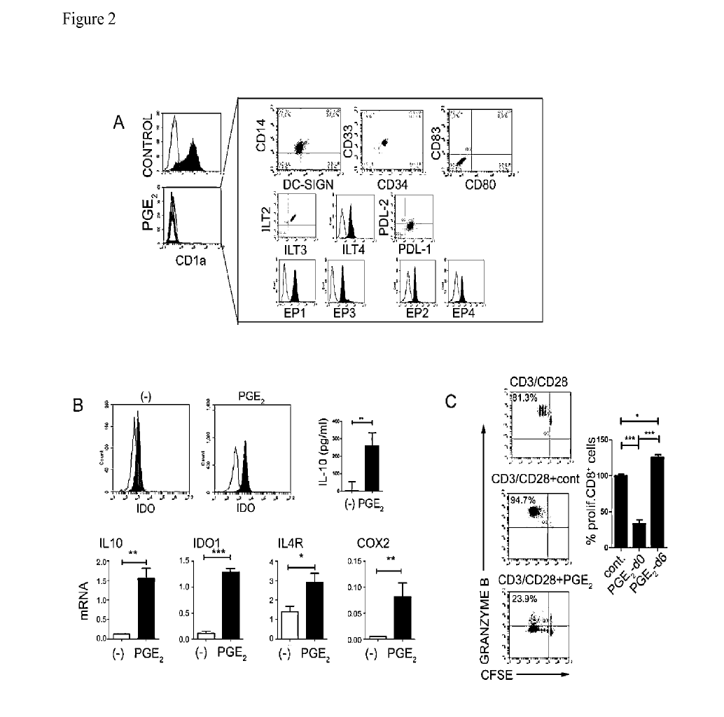

FIG. 3. PGE₂, EP4 and EP2 agonists mediate enhanced development of MDSCs. Induction of immunosuppressive factors by PGE₂, EP4 agonist (CAY10598), EP2 agonist (Butaprost), but not EP3/1 agonist (Sulprostone). All data were confirmed in 3-7 independent experiments. Bar graphs present data of a single representative experiment with different donors as mean±s.d.

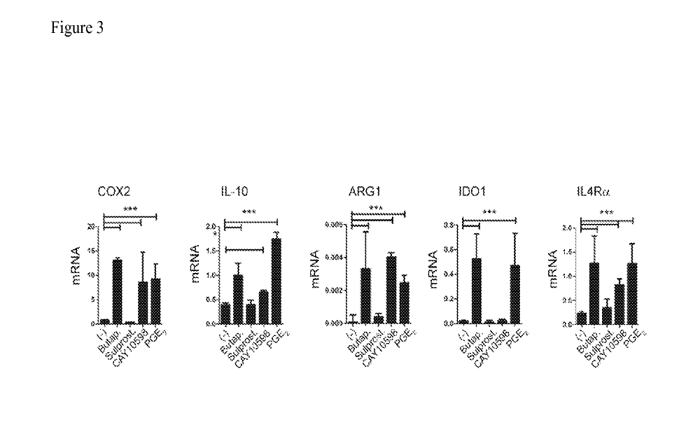

FIG. 4 Minimal requirement for high doses of PGE₂ in the functional induction of myeloid suppressive cells, regardless of the presence or absence of IL-4. (A) Dose-dependent expression of immunosuppressive factors IL10, IDO1, IL4Rα and COX2 in PGE₂-induced suppressive cells, generated in the presence or absence of IL-4. Histograms present data of a single representative experiment with different donors as mean±s.d.

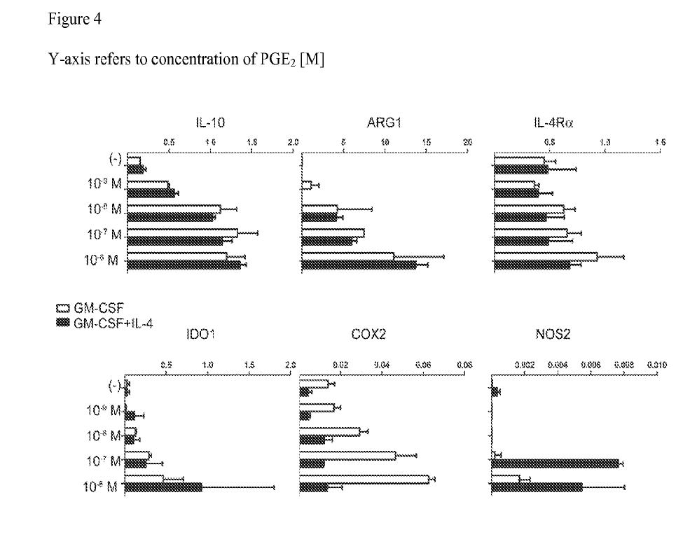

FIG. 5. PGE₂ allows for ex vivo generation of high numbers of suppressive cells. PGE₂ induces high numbers of suppressive cells (48.6%), with yields similar to iDCs (40.2%) and TNF-α matured DCs (36.9%). Histograms present data of individual experiments (N=12) with different donors as mean±s.d.

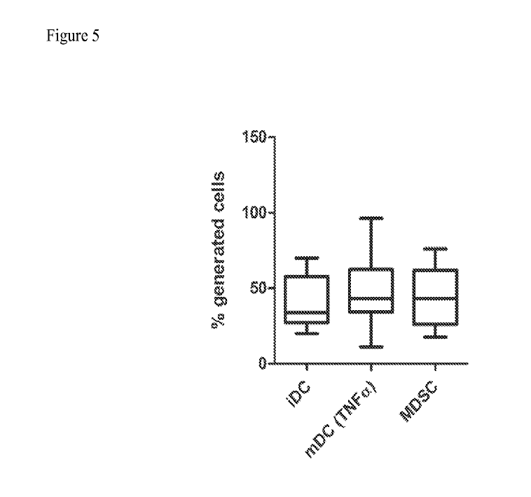

FIG. 6. Model: Positive COX2/PGE₂ feedback loop, induced by exogenous PGE₂, redirects DC differentiation towards suppressive cells. Low doses of PGE₂, allow for the induction of COX2, the inducer of endogenous PGE₂ production, in monocytic cells, associated with the induction of additional suppressive factors (i.e. IDO1, IL-10, ARG1, NOS2), and acquisition of suppressive functions. These processes are further amplified by the de novo-produced endogenous PGE₂, now produced at high levels by suppressive cells themselves, thereby creating a positive feedback loop. In addition to inducing other suppressive factors, PGE₂ also directly suppresses CTL development and functions, acting via EP2 and EP4 receptors.

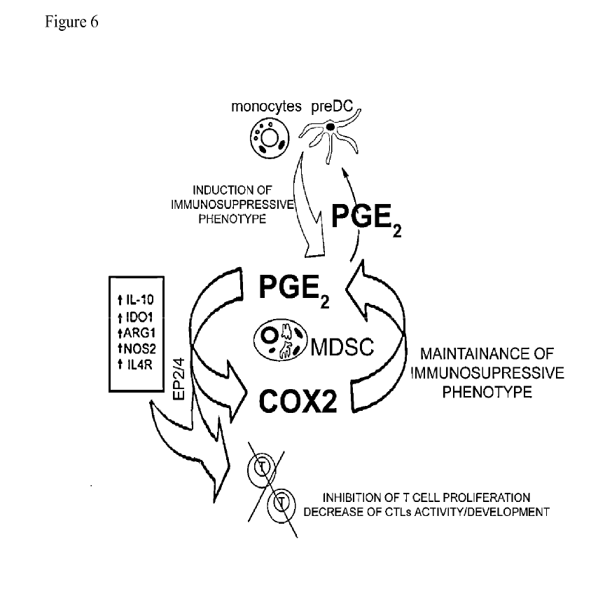

FIG. 7. Pathways of PGE₂ synthesis and PGE₂ signaling. PGE₂ synthesis involves phospholipase A2 (PLA2), cyclooxygenases (COX1 and COX2) that convert arachidonic acid into prostaglandin H₂ (PGH₂), and prostaglandin E synthases (PGES), responsible for the final synthesis of PGE₂. The rate of PGE₂ degradation in vivo in individual tissues is controlled by 15-hydroxyprostaglandin dehydrogenase (15-PGDH). The heterogeneous effects of PGE₂ are reflected by the existence of four different PGE₂ receptors, designated EP1, EP2, EP3 and EP4, with an additional level of functional diversity resulting from multiple splice variants of EP3 that exists in at least 8 forms in humans and 3 forms in mice. The signaling through the two Gₛ-coupled receptors, EP2 and EP4, is mediated by the adenylate cyclase-triggered cAMP/PKA/CREB pathway, mediating the dominant aspects of the anti-inflammatory and suppressive activity of PGE₂. While EP2 is believed to signal in a largely cAMP-dependent fashion, EP4 also activates the PI3K-dependent ERK1/2 pathway. However, both EP2 and EP4 have been shown to activate the GSK3/β-catenin pathway. EP1 and high affinity EP3 are not coupled to Gₛ and lack cAMP-activating functions. Most of the splice variants of EP3 represent Gᵢ-coupled PGE₂ receptors capable of inhibiting cAMP, although at least some of them can also exist in a Gₛ-coupled form capable of cAMP activation, with different sensitivities to ligand-induced desensitization³⁶. The mode of signaling via EP1 remains unclear, but involves calcium release.

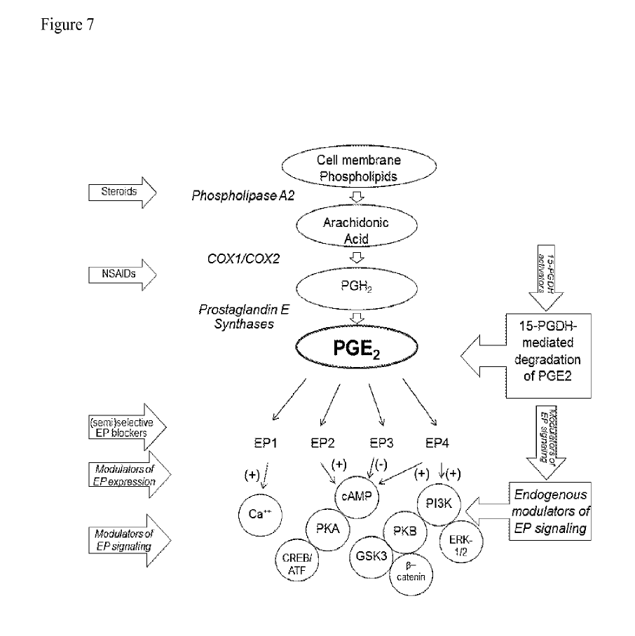

1. A method for treating or preventing the onset or recurrence of an autoimmune disease, chronic inflammatory disease, transplant rejection, or GvH, comprising administering to the subject therapeutically effective amounts of myeloid cells pretreated with a therapeutically effective amount of an activator of the cAMP-signaling pathway, adelylate cyclase, a phosphodiesterase inhibitor, CREB a downstream element of the CREB signaling pathway, or combinations thereof.

2. The method of claim 1 wherein the activator of the c-AMP signaling pathway is a prostaglandin or a prostaglandin analog.

3. The method of claim 2 wherein the prostaglandin or prostaglandin analog activates EP2 and/or EP4 receptors.

4. (canceled)

5. The method of claim 1, wherein the activator of the cAMP-signaling pathway is combined with an effective amount of GM-CSF, IL-4, or a combination thereof.

6-8. (canceled)

9. The method of claim 1, where the myeloid cells are monocytes.

10. The method of claim 1, where the myeloid cells are myeloid cells generated ex vivo from bone marrow-isolated or blood-isolated precursor cells.

11-13. (canceled)

14. The method of claim 1, wherein an implantable bioreactor or implantable system is used to prepare the myeloid cells in vivo.

15. A method for treating or preventing the onset or recurrence of an autoimmune disease, chronic inflammatory disease, transplant rejection, or GvH, comprising administering to the subject therapeutically effective amounts of a) a prostaglandin or other activator of the cAMP-signaling pathway and b) a chemokine, chemokine-inducing factor or alternative attractant of myeloid cells.

16-18. (canceled)

19. The method of claim 15, wherein the chemokine is CCL2, CCL3, CCL4, CCL5, CCL6, CCL7, CCL8, CCL12, CCL13, CL15, CCL16, CCL20, CCL23, CXCL14 or CX3CL1.

20-22. (canceled)

23. The method of claim 15, wherein the said chemokine-inducing factor is a proinflammatory cytokine or a TLR-ligand.

24-34. (canceled)
